(19) United States
(12) Patent Application Publication
(10) Pub. No.: US 2025/0078968 A1
(43) Pub. Date: Mar. 6, 2025

(54) STORAGE MEDIUM, TERMINAL DEVICE, AND MEDICAL INFORMATION PROPOSAL DEVICE

(71) Applicant: CANON MEDICAL SYSTEMS CORPORATION, Otawara-shi (JP)

(72) Inventors: Hayato OKUMIYA, Nasushiobara (JP); Atsuko SUGIYAMA, Nasushiobara (JP)

(73) Assignee: CANON MEDICAL SYSTEMS CORPORATION, Otawara-shi (JP)

(21) Appl. No.: 18/815,810

(22) Filed: Aug. 26, 2024

(30) Foreign Application Priority Data
Aug. 28, 2023 (JP) 2023-138426

(51) Int. Cl. G16H 10/60; G16H 15/00

(52) U.S. Cl. CPC G16H 10/60; G16H 15/00

(57) ABSTRACT

According to an embodiment, a computer-readable non-transitory storage medium stores a program for causing a computer of a user to receive an input of disease information from the user, access medical data about the user stored in a storage medium separated from a medical institution, pick up medical data from the medical data stored, which is to be provided to the medical institution by the user in accordance with the disease information, and propose the picked medical data to the user before the picked medical data is provided to the medical institution.

### CROSS-REFERENCE TO RELATED APPLICATION

Priority is claimed on Japanese Patent Application No. 2023-138426, filed Aug. 28, 2023, the content of which is incorporated herein by reference.

### FIELD

Embodiments disclosed in the present specification and the drawings relate to a storage medium, a terminal device, and a medical information proposal device.

### BACKGROUND

In recent years, there have been cases in which medical information about many people is managed by a business operator (hereinafter referred to as a medical information manager) for professionally managing medical information about personal health for use in medical care. The medical information includes, for example, medical examination and treatment data such as an electronic medical record (EMR) and an electronic health record (EHR) managed by medical institutions, a personal health record (PHR) including personal health data, and the like.

For example, the medical information manager provides medical information for managing patients to medical institutions for medically examining and treating the patients, thereby contributing to rapid and highly effective medical examination and treatment. The medical information, for example, may be provided to medical institutions directly from the medical information manager or to medical institutions through patients.

For example, when there is a patient who wants to be medically examined and treated by a doctor due to the onset of symptoms of a disease, medical information is provided to a medical institution before the patient is medically examined in the medical institution, such that the medical examination and treatment are performed smoothly and efficiently. However, it is difficult for patients to recognize medical information useful for the medical examination and treatment. Furthermore, because the medical information includes so-called personal information, the medical information may include information that a patient does not want others to know or information that a patient does not want to provide to others.

### BRIEF DESCRIPTION OF THE DRAWINGS

FIG. 1 is a diagram showing an example of a configuration of a medical information proposal system of an embodiment.

FIG. 2 is a diagram showing lists of life information and medical history information.

FIG. 3 is a sequence diagram showing an example of a flow of a process in the medical information proposal system.

FIG. 4 is a diagram showing an example of an interface screen displayed on a touch panel.

FIG. 5 is a diagram showing an example of an interface screen displayed on the touch panel.

FIG. 6 is a diagram showing an example of an interface screen displayed on the touch panel.

FIG. 7 is a flowchart showing an example of a flow of a process in a medical institution.

### DETAILED DESCRIPTION

Hereinafter, a storage medium, a terminal device, and a medical information proposal device of embodiments will be described with reference to the drawings. According to an embodiment, there is provided a computer-readable non-transitory storage medium storing a program for causing a computer of a user to receive an input of disease information from the user, access medical data about the user stored in a storage medium separated from a medical institution, pick up medical data from the medical data stored, which is to be provided to the medical institution by the user in accordance with the disease information, and propose the picked medical data to the user before the picked medical data is provided to the medical institution. Thereby, appropriate information can be provided to the medical institution.

In this embodiment, the chief compliment or chief compliment information are one example of disease information input by a user. The chief compliment represents abnormality which the user feels, disease type, or disease situation. The chief compliment doesn't have to represent only one disease, it can comprise multiple entry about type, situation, or abnormality at a time.

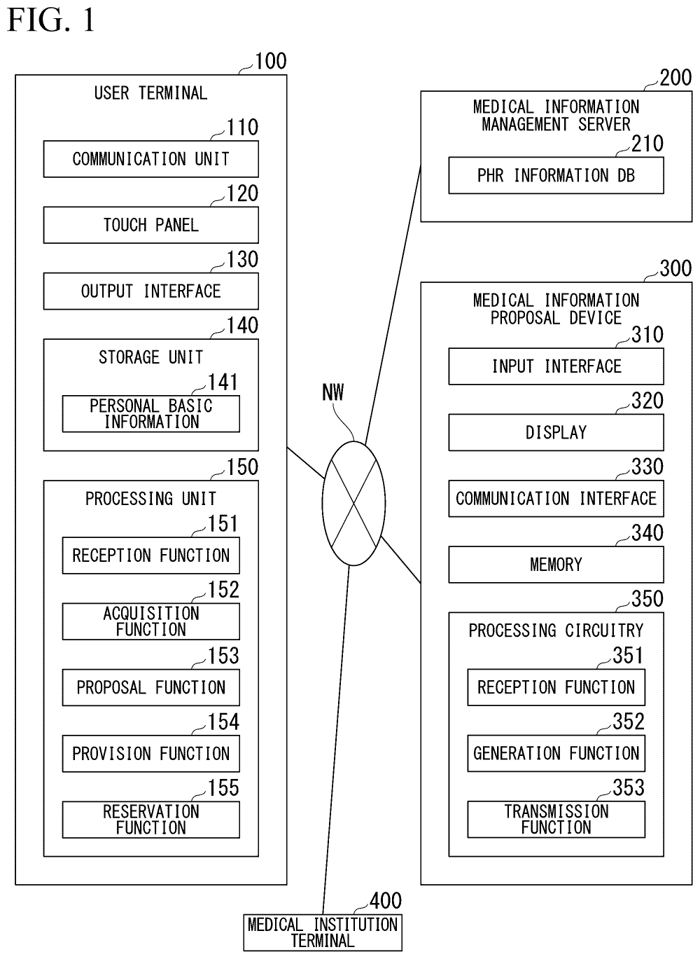

FIG. 1 is a diagram showing an example of a configuration of a medical information proposal system 1 of an embodiment. The medical information proposal system 1 includes, for example, a user terminal 100, a medical information management server 200, a medical information proposal device 300, and a medical institution terminal 400. The medical information proposal system 1 of the embodiment, for example, is used in a medical institution reservation system for searching for a medical institution to be used and sending a reservation request to the found medical institution when a user who becomes a patient of a medical institution feels symptoms of a disease.

A medical institution reservation application for use in the medical institution reservation system is installed on the user terminal 100. The user searches for a medical institution where the user receives a medical examination and treatment through the medical institution reservation system. The medical information proposal system 1 provides the user with proposal information (hereinafter referred to as recommendation information) as a proposal on which PHR data should be provided when the user provides medical data, for example, PHR data, to a medical institution.

The recommendation information is information in which PHR data is organized as information mainly for general practitioners or the like to perform medical examination based on the patient's chief complaint on the basis of guidelines, textbooks, and the like. The PHR data is an example of medical data. For example, when the medical institution reservation application is activated, the user terminal 100 transmits chief complaint information or the like to the medical information proposal device 300 and requests recommendation information.

The medical information proposal device 300 generates the recommendation information including the PHR data to be provided to the medical institution (hereinafter referred to as provision target PHR data) with reference to the PHR information database (hereinafter referred to as DB) 210 or the like stored in the medical information management server 200 on the basis of the chief complaint information transmitted by the user terminal 100. The PHR information DB is an example of a provision target database.

The user, who is a patient to be medically examined in a medical institution, recognizes the provision target PHR data on the basis of the recommendation information proposed by the user terminal 100. The user performs an input manipulation for designating PHR data authorized to be provided to the medical institution (hereinafter referred to as authorized-provision-related PHR data) from the provision target PHR data.

The user terminal 100 receives the recommendation information transmitted by the medical information proposal device 300 and proposes the provision target PHR data included in the recommendation information to the user. The user designates PHR data, which may be provided to a medical institution from among the provision target PHR data, as the authorized-provision-related PHR data.

On the other hand, the user terminal 100 presents a medical institution list to the user on the basis of the symptoms of the disease, the user's address or current location, and the like. The user selects a medical institution at which to receive a medical examination from presented medical institutions. The user terminal 100 generates reservation request information for the medical institution terminal 400 of the medical institution selected by the user. The user terminal 100 transmits the authorized-provision-related PHR data designated by the user to the medical institution terminal 400 of the medical institution together with the reservation request information.

The medical institution makes a reservation for a medical examination for the user on the basis of the reservation request information transmitted to the medical institution terminal 400 by the user terminal 100. The authorized-provision-related PHR data transmitted to the medical institution terminal 400 is received and the medical examination for the user is prepared. For example, the user is medically examined on a date and time of a reservation. The user terminal 100, for example, may execute a medical institution reservation program provided by the medical information proposal device 300 with a browser, instead of a medical institution reservation application. The medical institution reservation application and the medical institution reservation program are both examples of programs. Although the medical institution reservation application is used in the user terminal 100 in the embodiment, a medical information proposal application or a medical information proposal program for proposing medical information without searching for a medical institution or making a reservation may be used instead of the medical institution reservation application or the medical institution reservation program.

### [User Terminal 100]

The user terminal 100 is a terminal device managed by the user. The user terminal 100 is, for example, a portable terminal device such as a smartphone or a tablet terminal. The user terminal 100 may be, for example, an on-site or portable personal computer other than a portable terminal device.

The user terminal 100 includes, for example, a communication unit 110, a touch panel 120, an output interface 130, a storage unit 140, and a processing unit 150. The communication unit 110, for example, communicates with external devices such as a medical information management server 200, a medical information proposal device 300, and a medical institution terminal 400 via a network NW such as a local area network (LAN). The communication unit 110 includes, for example, a communication interface such as a network interface card (NIC). The network NW may include the Internet, a cellular network, a Wi-Fi network, a wide area network (WAN), or the like instead of or in addition to the LAN.

The touch panel 120, for example, functions as an input interface to which the user inputs information and an output interface configured to display the input information and the like. The touch panel 120 displays, for example, a graphical user interface (GUI) for the user to perform various types of input manipulations and the like. The touch panel 120 outputs information corresponding to the input manipulation from the user to the processing unit 150.

The output interface 130 includes, for example, a speaker, a vibrator, and the like. The speaker outputs a sound in accordance with control of the processing unit 150. The vibrator vibrates in accordance with the control of the processing unit 150.

The storage unit 140 is implemented by, for example, a semiconductor memory element such as a random-access memory (RAM) or a flash memory. The storage unit 140 may include a non-transitory storage medium such as a read-only memory (ROM) or a register. The storage unit 140 stores personal basic information 141. The personal basic information 141 includes personal basic information about the user, for example, a user ID for identifying the user, the user's name, age, gender, height, and weight, and the like.

The processing unit 150 is implemented by, for example, a hardware processor such as a central processing unit (CPU) executing a program (software) such as a medical institution reservation application. The processing unit 150 has, for example, a reception function 151, an acquisition function 152, a proposal function 153, a provision function 154, and a reservation function 155.

The reception function 151 is performed to receive a designation input corresponding to the input manipulation of the user on the touch panel 120. For example, the user inputs information about a chief complaint corresponding to his or her symptoms by manipulating the touch panel 120. The touch panel 120 outputs information about the chief complaint input by the user's manipulation to the processing unit 150. The touch panel 120 is an example of an input unit.

The processing unit 150 receives an input of the chief complaint from the user on the basis of the information about the chief complaint output by the touch panel 120 with the reception function 151. When the input of the chief complaint is received from the user, the reception function 151 is performed to generate request information for requesting the chief complaint information about the content of the chief complaint and the provision target PHR data corresponding to the chief complaint. The reception function 151 is performed to transmit the generated request information and the chief complaint information to the medical information proposal device 300 together with the user ID included in the personal basic information 141 stored in the storage unit 140. The reception function 151 is an example of a reception unit.

When the request information is transmitted by the user terminal 100, the medical information proposal device 300 generates recommendation information including the provision target PHR data corresponding to the chief complaint indicated in the chief complaint information and transmits the recommendation information to the user terminal 100. The recommendation information includes one or more provision target PHR data items. The provision target PHR data included in the recommendation information includes PHR data of the user and PHR data other than the PHR data of the user.

The acquisition function 152 is performed to acquire recommendation information transmitted by the medical information proposal device 300. The acquisition function 152 is an example of an acquisition unit. PHR data other than the PHR data of the user is not transmitted to the medical information proposal device 300 and may be transmitted to a terminal other than the medical information proposal device 300, for example, the user terminal 100 or the medical institution terminal 400.

The proposal function 153 is performed to make a proposal by causing the touch panel 120 to display the provision target PHR data included in the recommendation information or causing the speaker provided in the output interface 130 to output a sound and performing an output process for the user. The proposal function 153 is an example of a proposal unit. The touch panel 120 and the output interface 130 are examples of an output unit. In accordance with the function of the proposal function 153, the user performs a manipulation for designating the authorized-provision-related PHR data authorized to be provided to a medical institution from among the provision target PHR data and inputting the designated PHR data to the touch panel 120. The reception function 151 is performed to further receive an input of authorized-provision-related PHR data authorized to be provided by the user on the basis of the user's input manipulation.

The provision function 154 is performed to receive an input manipulation from the user and decide on the authorized-provision-related PHR data. The authorized-provision-related PHR data may be PHR data designated by the user from PHR data of the user or PHR data designated by the user from all of the PHR data of the user and PHR data other than the PHR data of the user. The provision function 154 is performed to transmit the generated authorized-provision-related PHR data to the medical institution terminal 400 together with the chief complaint, thereby providing the medical institution therewith. The provision function 154 is an example of a provision unit.

The reservation function 155 is performed to present the user with a candidate medical institution where the user will be medically examined on the basis of the user's chief complaint, current location, address, and the like. The user designates a medical institution where the user will be medically examined among presented candidates, a date and time of a reservation in the designated medical institution, and the like. The reservation function 155 is performed to generate reservation request information based on the date and time of the reservation in the designated medical institution. The reservation function 155 is performed to include chief complaint information in the generated reservation request information and transmit the reservation request information to the medical institution terminal 400 of the medical institution designated by the user.

### [Medical Information Management Server 200]

The medical information management server 200 includes, for example, a PHR information DB 210. The medical information management server 200 creates a PHR data list including a part or all of the PHR data included in the stored PHR information DB 210 on the basis of the list request information transmitted by the medical information proposal device 300 and provides the PHR data list by transmitting the PHR data list to the medical information proposal device 300. The medical information management server 200 may store list request information transmitted by the medical information proposal device 300.

The medical information management server 200 may provide a PHR data list by transmitting the PHR data list to the user terminal 100 or the medical institution terminal 400 other than the medical information proposal device 300. The medical information management server 200 may provide the PHR data list by transmitting the PHR data list to two or more of the user terminal 100, the medical information proposal device 300, and the medical institution terminal 400.

The PHR information DB 210 is, for example, a database in which PHR data including PHRs of a plurality of users is collected. The PHR data is, for example, a DB of a list of items widely and generally examined by a general practitioner. The PHR data includes EHR data and the EHR data includes EMR data. A user ID is attached to the PHR data and information about a user (a patient) corresponding to the PHR data can be identified.

The PHR data includes, for example, medical data (including EHR data, EMR data, and prescription data), health checkup data, daily life log data (a GPS location and home measurement content such as eating, drinking, and walking), for example, life information about the user's life and medical history information about a medical history. The medical history information includes, for example, data related to previous medical examination and treatment results, for example, a history of previous medical examination and treatment results.

PHR and EHR DBs (not shown), for example, may be managed by a distributed, centralized, or blockchain DB configuration or may be managed by a physical policy for on-premise or cloud. The medical information management server 200 may be managed by, for example, various management entities such as countries (related ministries and agencies and public organizations), companies, and individuals.

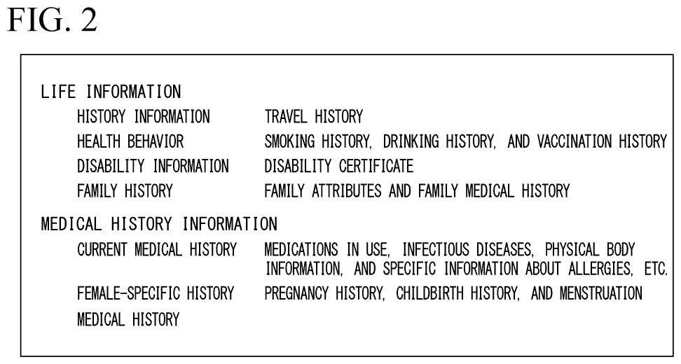

FIG. 2 is a diagram showing lists of life information and medical history information. The life information includes, for example, history information, health behavior, disability information, and various types of information about a family history. The history information includes, for example, information about the user's travel history and the like. The health behavior includes, for example, information about the user's smoking history, drinking history, and vaccination history, and the like. The disability information includes, for example, information about the presence or absence of a disability certificate and the like. The family history includes, for example, information about the user's family attributes and family medical history and the like.

The medical history information includes, for example, various types of information about the current medical history, the female-specific history, and the user's medical history. The current medical history includes, for example, information of medications in use, infectious diseases, physical body information, and specific information about allergies and the like. The female-specific history includes, for example, information about a pregnancy history, a childbirth history, and menstruation. The medical history includes information about an example of a medical history of the user.

### [Medical Information Proposal Device 300]

The medical information proposal device 300 includes, for example, an input interface 310, a display 320, a communication interface 330, a memory 340, and processing circuitry 350. The input interface 310 receives various types of input manipulations of a manipulator and outputs an electrical signal indicating content of the received input manipulation to the processing circuitry 350.

The input interface 310 is implemented, for example, by a mouse, keyboard, touch panel, drag ball, switch, button, joystick, camera, infrared sensor, microphone, and the like. The input interface 310 may be implemented by a display device (e.g., a tablet terminal) that can communicate wirelessly with a main unit.

The display 320 displays various types of information. The display 320 is, for example, a liquid crystal display, a cathode ray tube (CRT), an organic electroluminescence (EL) display, or the like. The display 320 may be a desktop type device or a display device (e.g., a tablet terminal) capable of wireless communication with the main unit.

The communication interface 330, for example, communicates with an external device such as the user terminal 100, the medical information management server 200, and the medical institution terminal 400 via the network NW such as a LAN. The communication interface 330 includes, for example, a communication interface such as an NIC. The network NW may include the Internet, cellular network, Wi-Fi network, WAN, and the like instead of or in addition to the LAN.

The memory 340 is implemented by, for example, a semiconductor memory element such as a RAM or a flash memory and a non-transitory storage medium such as a hard disk or an optical disc. These non-transitory storage media may be implemented by other storage devices connected via a communication network such as a network attached storage (NAS) or an external storage server device. Moreover, the memory 340 may include a non-transitory storage medium such as a read-only memory (ROM) or a register.

The processing circuitry 350 includes, for example, a processor such as a CPU. For example, a reception function 351, a generation function 352, and a transmission function 353 are provided. The processing circuitry 350 implements these functions by, for example, executing a program stored in a storage device (storage circuitry) in a hardware processor.

A hardware processor includes, for example, is circuitry such as a CPU, a graphics processing unit (GPU), an application-specific integrated circuit (ASIC), or a programmable logic device (e.g., a simple programmable logic device (SPLD), a complex programmable logic device (CPLD), or a field programmable gate array (FPGA)). Instead of storing the program in the storage device, the program may be configured to be directly embedded in the circuitry of the hardware processor. In this case, the hardware processor implements each function by reading and executing the program embedded in the circuitry. The hardware processor is not limited to being configured as single circuitry and may be configured as one hardware processor by combining a plurality of pieces of independent circuitry to implement each function. The storage device may be a non-transitory (hardware) storage medium. Moreover, a plurality of components may be integrated into one hardware processor to implement each function.

Components of the processing circuitry 350 may be distributed and implemented by a plurality of pieces of hardware. The processing circuitry 350 may be implemented by a processing device capable of communicating with a main body. Each function included in the processing circuitry 350 may be distributed to a plurality of circuits or may be made available by activating the application software stored in the memory 340.

The reception function 351 is performed to receive the request information and the chief complaint information transmitted by the user terminal 100 using the communication interface 330. When the request information is received, the reception function 351 is performed to generate list request information with the user ID of the user who has transmitted the request information and transmit the list request information to the medical information management server 200. The medical information management server 200 transmits a PHR data list including PHR data with a user ID attached to the list request information transmitted by the medical information proposal device 300 and other PHR data to the medical information proposal device 300. The reception function 351 is an example of a reception unit.

The reception function 351 is performed to receive the PHR data list transmitted by the medical information management server 200. The generation function 352 is performed to select one or more provision target PHR data items corresponding to the user's chief complaint on the basis of a result of matching between the PHR data included in the PHR data list transmitted by the medical information management server 200 and the chief complaint indicated in the chief complaint information. Within the user's PHR data, only PHR data with the agreement of the user may be designated as a matching target with respect to the user's past medical examination and treatment results. When the medical institution publishes a reception/non-reception list, matching based on the reception/non-reception list may be performed.

The provision target PHR data includes, for example, prescription data related to prescriptions for the user, inspection data related to inspections in the medical examination performed on the user, medical examination and treatment facility data related to a specific medical examination and treatment facility, and home measurement data based on measurements at the user's home. The home measurement data includes, for example, data such as a body temperature, blood pressure, and heart rate in addition to a height and weight measured at the user's home or the like.

The generation function 352 is performed to set a priority level for the generated provision target PHR data. The generation function 352, for example, is performed to acquire a reference frequency at which the provision target PHR data has been referred to in the medical institution. The generation function 352 is performed to assign a higher priority level to the provision target PHR data when the acquired reference frequency is higher. For example, the generation function 352 is performed to acquire a ratio at which the user has provided the medical institution with the provision target PHR data as authorized-provision-related PHR data. The generation function 352 is performed to set a higher priority level when the ratio at which the user has provided the provision target PHR data is lower.

The generation function 352 is performed to generate recommendation information including the provision target PHR data with the priority level. The recommendation information includes all provision target PHR data generated by the generation function 352. The transmission function 353 is performed to transmit the recommendation information generated by the generation function 352 to the user terminal 100 using the communication interface 330. The generation function 352 is an example of a generation unit. The transmission function 353 is an example of a transmission unit.

The medical institution terminal 400 acquires reservation request information and authorized-provision-related PHR data provided by the user terminal 100. The medical institution terminal 400 makes a reservation for the user' medical examination on the basis of the provided reservation request information. In the medical institution in which the medical institution terminal 400 is provided, preparations for medically examining the user are started with reference to the chief complaint information included in the reservation request information and the provided authorized-provision-related PHR data. The medical institution terminal 400 uses the authorized-provision-related PHR data provided by the user terminal 100 before or at the start of the user's medical examination.

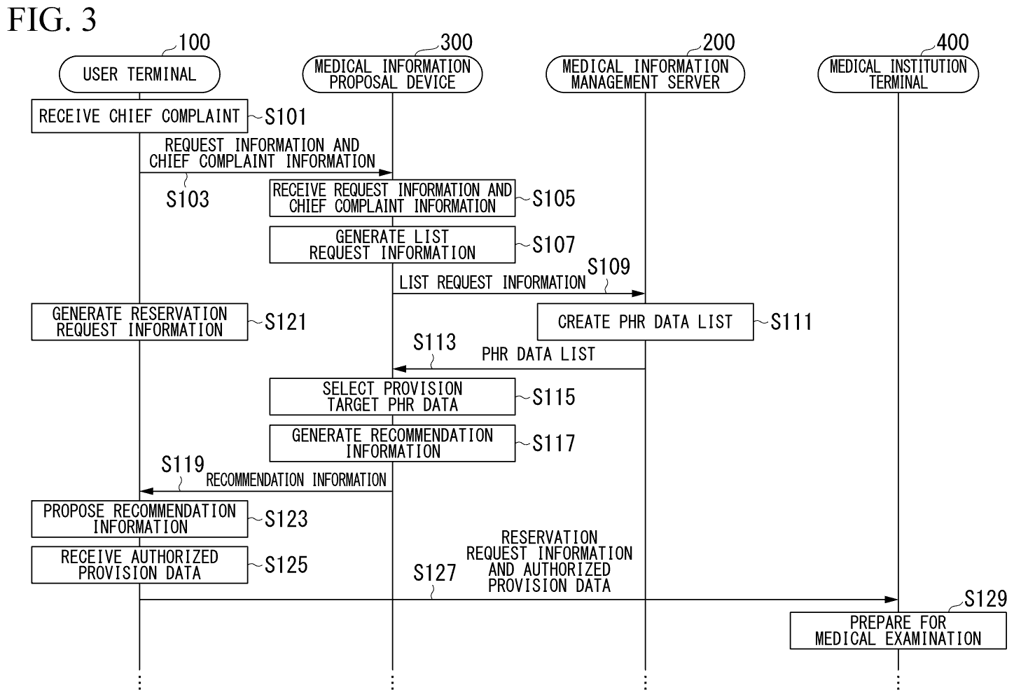

Next, a process of the medical information proposal system 1 will be described. FIG. 3 is a sequence diagram showing an example of a flow of a process in the medical information proposal system 1. The medical information proposal system 1 starts the process when the user is aware of symptoms of a disease, activates the medical institution reservation application of the user terminal 100, and inputs the chief complaint.

The user terminal 100 receives the chief complaint input by the user manipulating the touch panel 120 in the reception function 151 (step S101). The reception function 151 in which the chief complaint has been received is performed to generate request information, add a user ID thereto, and transmit the request information with the user ID to the medical information proposal device 300 together with chief complaint information (step S103).

The medical information proposal device 300 receives the request information and the chief complaint information transmitted by the user terminal 100 in the reception function 351 (step S105). The reception function 351 is performed to generate list request information on the basis of the received chief complaint information and the user ID (step S107). The reception function 351 is performed to transmit the generated list request information to the medical information management server 200 (step S109).

The medical information management server 200 creates a PHR data list on the basis of the list request information transmitted by the medical information proposal device 300 (step S111). The medical information management server 200 transmits the created PHR data list to the medical information proposal device 300 (step S113). The medical information proposal device 300 receives the PHR data list transmitted by the medical information management server 200 in the reception function 351.

Subsequently, the medical information proposal device 300 performs a matching process between the PHR data included in the PHR data list transmitted by the medical information management server 200 and the chief complaint indicated in the chief complaint information in the generation function 352. The generation function 352 is performed to select one or more provision target PHR data items corresponding to the user's chief complaint on the basis of a matching result (step S115).

When a plurality of provision target PHR data items are selected, the generation function 352 is performed to set a priority level for each provision target PHR data item. The generation function 352 is performed to generate recommendation information including the provision target PHR data with the priority level (step S117). The transmission function 353 is performed to transmit the recommendation information generated by the generation function 352 to the user terminal 100 (step S119).

Subsequently, in the proposal function 153, the user terminal 100 proposes recommendation information to the user by displaying the provision target PHR data included in the recommendation information transmitted by the medical information proposal device 300 on the touch panel 120 and the like (step S123). The user performs an input manipulation on the touch panel 120 so that the authorized-provision-related PHR data is designated within the provision target PHR data displayed on the touch panel 120.

The user terminal 100 receives an input of designation of the authorized-provision-related PHR data corresponding to the input manipulation of the user on the touch panel 120 in the reception function 151 (step S125). In the provision function 154, the user terminal 100 transmits and provides the authorized-provision-related PHR data (authorized provision data) corresponding to the input manipulation of the user to the medical institution terminal 400 together with the chief complaint information (step S127).

The medical institution terminal 400 makes a reservation for a medical examination on the user on the basis of the transmitted reservation request information. The medical institution equipped with the medical institution terminal 400 uses the chief complaint information and the authorized-provision-related PHR data included in the transmitted reservation request information to perform the preparation for the medical examination such as determining a medical examination and treatment policy when the user is medically examined and arranging necessary equipment (step S129). Thus, the process of the medical information proposal system 1 ends.

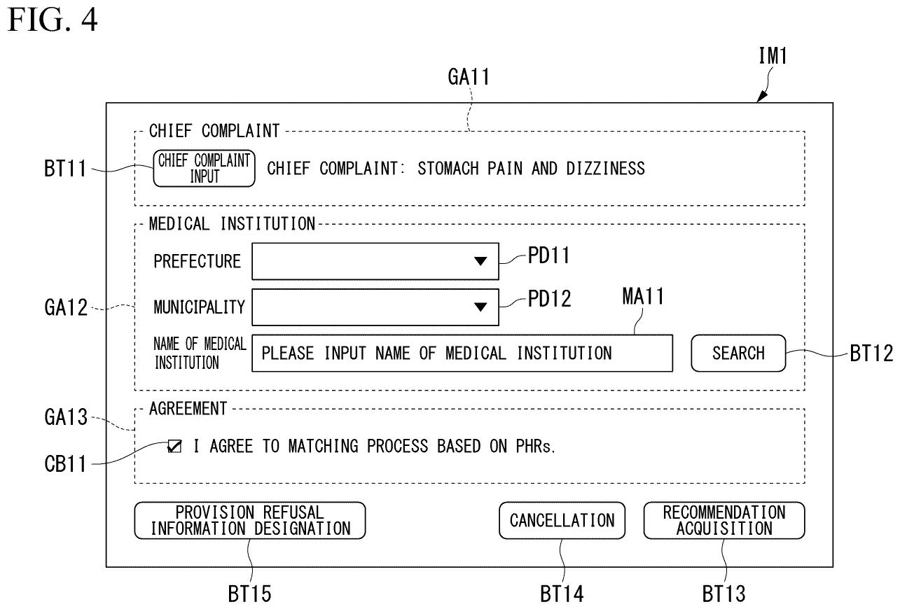

Next, a situation when the user performs an input manipulation on the user terminal 100 from the time when the user searches for a medical institution where the user will be medically examined and the time when the user transmits reservation request information and authorized-provision-related PHR data to the medical institution will be described. FIGS. 4 to 6 are diagrams showing examples of an interface screen displayed on the touch panel 120.

The user who feels symptoms of a disease manipulates the user terminal 100 to activate the medical institution reservation application. FIG. 4 shows a first interface screen IM1 displayed after the medical institution reservation application is activated. For example, a first display area GA11, a second display area GA12, and a third display area GA13 are displayed on the first interface screen IM1

The first display area GA11 is an area for displaying content related to the chief complaint. In the first display area GA11, the chief complaint input button BT11 and the content of a chief complaint (e.g., stomach pain and dizziness) are displayed. The user can input information about the chief complaint by manipulating the chief complaint input button BT11.

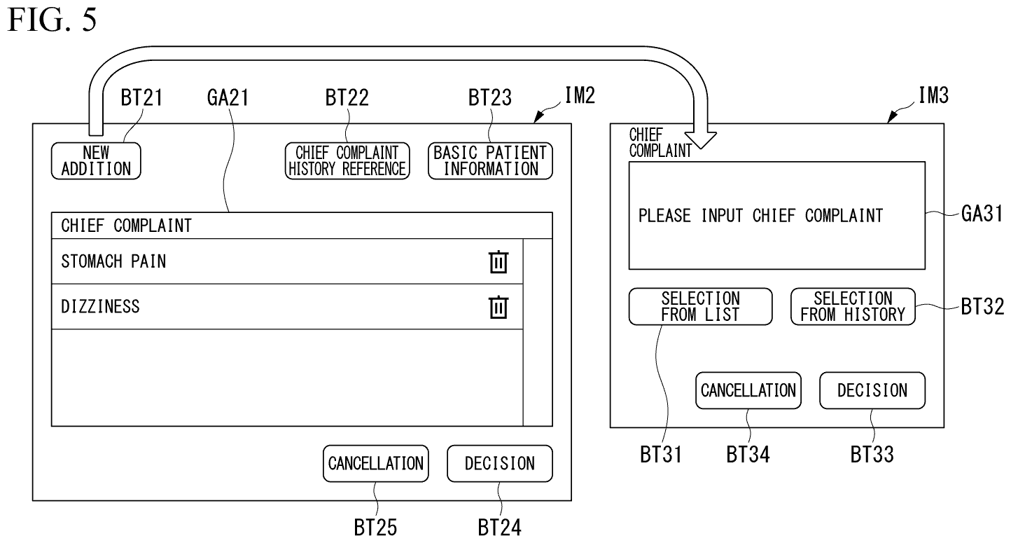

A second interface screen IM2 shown in the left of FIG. 5 is a screen displayed after the chief complaint input button BT11 is manipulated. On the second interface screen IM2, for example, a new addition button BT21, a chief complaint history reference button BT22, a basic patient information button BT23, a decision button BT24, a cancellation button BT25, and a condition information display area GA21 are displayed.

The new addition button BT21 is, for example, a button to be used when the user inputs a new chief complaint. When the new addition button BT21 is manipulated by the user or the like, a third interface screen IM3 shown in the right of FIG. 5 is displayed on the touch panel 120. On the third interface screen IM3, for example, a symptom input area GA31, a list display button BT31, a history display button BT32, a decision button BT33, and a cancellation button BT34 are displayed.

In the symptom input area GA31, content of symptoms including the user's chief complaints, for example, chief complaints such as “stomach pain” and “dizziness” and symptoms such as “getting hungry easily” and “not sleeping well” can be input. In the symptom input area GA31, for example, symptoms freely written by the user may be displayed, or symptoms selected by the user from pre-prepared options may be displayed.

The list display button BT31 is a button for displaying content of a list of general chief complaints. The chief complaint list includes, for example, a plurality of specific content items that can be chief complaints, such as “headache,” “stomach pain,” “dizziness,” “palpitations,” and “leg injury.” The user can select and designate a current chief complaint from the displayed list of chief complaints.

The history display button BT32 is a button for displaying a list of chief complaints previously designated by the user. In the list, the previously designated chief complaints are listed, for example, in order from the new chief complaint (the most recently designated chief complaint). The user can select and designate a current chief complaint from the displayed list of previously designated chief complaints.

The decision button BT33 is a button for deciding on the chief complaint input to the symptom input area GA31 and switching the display of the touch panel 120 from the third interface screen IM3 to the second interface screen IM2. The cancellation button BT34 is a button for switching the display of the touch panel 120 from the third interface screen IM3 to the second interface screen IM2 without designating the chief complaint, regardless of whether or not the chief complaint has been input to the symptom input area GA31.

The chief complaint history reference button BT22 in the second interface screen IM2 is a button for displaying content of a plurality of chief complaints previously designated by the user. When the chief complaint history reference button BT22 is manipulated, the content of the chief complaints previously made by the user is displayed. The user can decide on the current chief complaint with reference to the displayed chief complaints.

The basic patient information button BT23 is a button for displaying the content of the patient's personal basic information 141. The decision button BT24 is a button for deciding on the designated chief complaint and switching the screen of the touch panel 120 from the second interface screen IM2 to the first interface screen IM1 shown in FIG. 4. The cancellation button BT25 is a button for canceling the designated chief complaint and switching the screen of the touch panel 120 from the second interface screen IM2 to the first interface screen IM1.

Returning to FIG. 4, the second display area GA12 is an area for displaying content for designating conditions when the user searches for a desired medical institution for a medical examination and treatment. In the second display area GA12, a prefecture selection field PD11, a municipality selection field PD12, a medical institution name input field MA11, and a search button BT12 are displayed.

The user designates a prefecture or municipality by manipulating a pull-down menu displayed in the prefecture selection field PD11 and the municipality selection field PD12 or inputs a name of a medical institution to the medical institution name input field MA11 to search for the desired medical institution for the medical examination and treatment. After a manipulation such as an input for the prefecture selection field PD11, the municipality selection field PD12, and the medical institution name input field MA11, the search button BT12 is manipulated and therefore a medical institution satisfying a condition is found.

The third display area GA13 is an area where the user displays content related to the user's agreement to the matching process. In the third display area GA13, a check box CB11 is displayed on the left side with the message “I agree to a matching process based on PHRs.” When the check box CB11 is manipulated and a check mark is displayed, the user agrees to a matching process for his or her PHR data.

A recommendation acquisition button BT13, a cancellation button BT14, and a provision refusal information designation button BT15 are displayed outside of the first display area GA11, the second display area GA12, and the third display area GA13. When the recommendation acquisition button BT13 is manipulated in a state in which the information about the chief complaint is input, the user terminal 100 generates request information and transmits the request information to the medical information proposal device 300 together with a user ID and chief complaint information.

When the cancellation button BT14 is manipulated, an input related to a reservation of a medical institution is canceled and the medical institution reservation application is terminated. When the provision refusal information designation button BT15 is manipulated, it is possible to designate PHR data that is not desired to be provided to a medical institution within the PHR data included in the recommendation information proposed by the medical information proposal device 300.

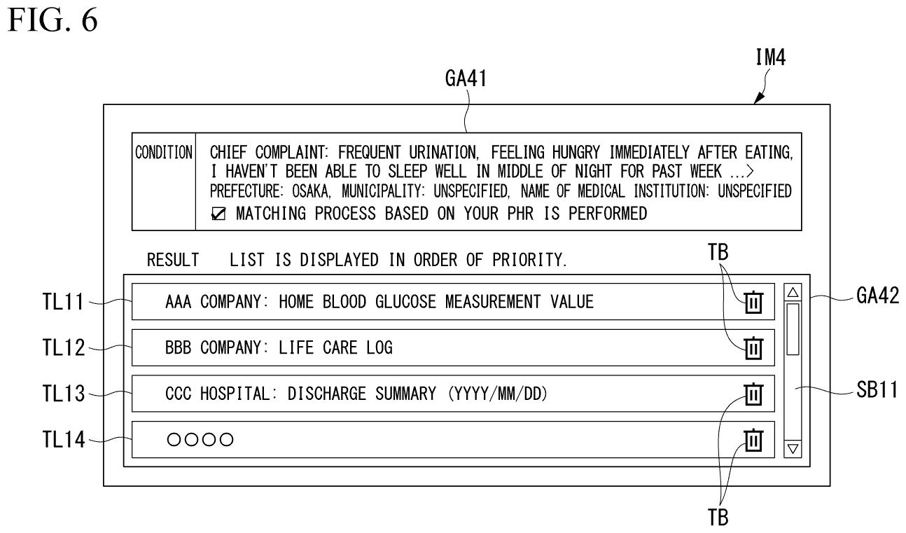

In FIG. 6, a fourth interface screen IM4 displayed on the touch panel 120 after the provision refusal information designation button BT15 is manipulated by the user is shown. On the fourth interface screen IM4, for example, a condition information display area GA41 and a recommendation information display area GA42 are displayed.

In the condition information display area GA41, for example, a condition designated on the first interface screen IM1 is displayed. In the condition information display area GA41, content of a chief complaint, a prefecture and municipality where a desired medical institution for the medical examination is located, the name of the medical institution, and information about whether or not a matching process with PHRs is possible are displayed. Information that has not been designated within the above-described information is displayed as unspecified.

For example, a first title display field TL11, a second title display field TL12, a third title display field TL13, a fourth title display field TL14, and a scroll bar SB11 are displayed in the recommendation information display area GA42. In the first to fourth title display fields TL11 to TL14, a title obtained by summarizing the provision target PHR data included in the recommendation information proposed to the medical information proposal device 300 under various conditions is displayed. The first to fourth title display fields TL11 to TL14 are displayed in order from the provision target PHR data with the highest priority level (priority).

For example, the title “AAA company: home blood glucose measurement value” is displayed in the first title display field TL11. Here, the provision target PHR data is PHR data of blood glucose measurement values measured at home by the user using a measurement device provided by AAA company. Likewise, the title “BBB company: life care log” is displayed in the second title display field TL12, and the title “CCC hospital: discharge summary (YYYY/MM/DD)” is displayed in the third title display field TL13.

On the right side of the first to fourth title display fields TL11 to TL14, the scroll bar SB11 is displayed. By manipulating the scroll bar SB11, the user can display another title display field. On the right side of the first to fourth title display fields TL11 to TL14, a trash box icon TB is displayed.

When the user manipulates the trash box icon TB, the title display field including the trash box icon TB that has been manipulated is erased. The provision target PHR data of the title for which the title display field has been erased is PHR data refused to be provided to a medical institution by the user. PHR data other than PHR data refused to be provided by the user within the provision target PHR data becomes authorized-provision-related PHR data. In the embodiment, after the PHR data that is refused to be provided is designated from the provision target PHR data, the remaining provision target PHR data is designated as authorized-provision-related PHR data. However, the authorized-provision-related PHR data may be designated from the provision target PHR data.

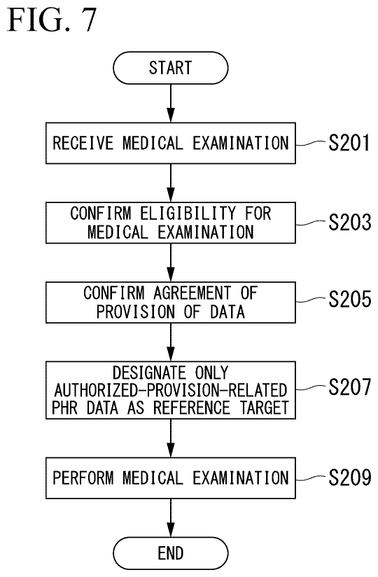

A flow on the day of a medical examination in a medical institution equipped with the medical institution terminal 400 for which the reservation for the user has been made on the basis of the reservation request information will be described. FIG. 7 is a flowchart showing an example of a flow of a process at a medical institution. On the day of the medical examination, the user visits the medical institution and is medically examined.

In the medical institution that the user has visited, the user receives a medical examination (step S201). When the user receives the medical examination, the medical institution confirms the eligibility for the medical examination by confirming the user's My Number Card (step S203). Subsequently, the medical institution confirms that the user has agreed to provide PHR data orally or the like (step S205).

Once the agreement of the user is confirmed, the medical institution confirms authorized-provision-related PHR data transmitted to the medical institution terminal 400. In the medical institution, only the authorized-provision-related PHR data whose provision is authorized by the user within provision target PHR data proposed to the user becomes a reference target (step S207). Subsequently, in the medical institution, the medical examination is performed with reference to the authorized-provision-related PHR data (step S209).

In the medical institutions, an electronic medical record is created at the time of the medical examination of the user. For example, the medical institution transmits the created electronic medical record to the medical information proposal device 300 with the medical institution terminal 400. The medical information proposal device 300 updates medical data included in the PHR information DB 210 on the basis of the transmitted electronic medical record.

According to at least one of the above-described embodiments, the user's computer can be allowed to perform a process of receiving an input of the user's chief complaint, acquiring medical data that the user should provide to a medical institution in accordance with the chief complaint, and proposing the medical data to the user, thereby making it possible to provide appropriate information to the medical institution.

While several embodiments of the present invention have been described above, these embodiments have been presented by way of example only, and are not intended to limit the scope of the inventions. These embodiments may be embodied in a variety of other forms. Various omissions, substitutions, and combinations may be made without departing from the spirit of the inventions. The inventions described in the accompanying claims and their equivalents are intended to cover such embodiments or modifications as would fall within the scope and spirit of the inventions.

What is claimed is:

1. A computer-readable non-transitory storage medium storing a program for causing a computer of a user to:
receive an input of disease information from the user;
access medical data about the user stored in a storage medium separated from a medical institution;
pick up medical data from the medical data stored, which is to be provided to the medical institution by the user in accordance with the disease information; and
propose the picked medical data to the user before the picked medical data is provided to the medical institution.

2. The computer-readable non-transitory storage medium according to claim 1, wherein the computer-readable non-transitory storage medium stores the program for causing the computer to further:
receive an input manipulation from the user designating an authorized-provision-related personal health record (PHR) authorized to be provided to the medical institution within the medical data; and
provide the authorized-provision-related PHR to the medical institution together with the disease information.

3. The computer-readable non-transitory storage medium according to claim 2, wherein the computer-readable non-transitory storage medium stores the program for causing the computer to further include data related to previous medical examination and treatment results of the user in the medical data and provide the medical data to the medical institution.

4. The computer-readable non-transitory storage medium according to claim 3, wherein the computer-readable non-transitory storage medium stores the program for causing the computer to further receive an input of the medical data to be provided to the medical institution from the user.

5. The computer-readable non-transitory storage medium according to claim 3, wherein the medical data includes at least one item of life information about the life of the user and medical history information about a medical history of the user.

6. The computer-readable non-transitory storage medium according to claim 1, wherein a priority level for provision to the medical institution is assigned to the medical data.

7. The computer-readable non-transitory storage medium according to claim 6, wherein the priority level is set to a higher level when a reference frequency for the disease information is higher.

8. The computer-readable non-transitory storage medium according to claim 6, wherein the priority level is set to a lower level when a ratio of provision when a medical examination and treatment of the disease information are performed is higher.

9. The computer-readable non-transitory storage medium according to claim 1, wherein the medical data includes at least one item of prescription data related to prescriptions, inspection data related to inspections, medical examination and treatment facility data related to a specific medical examination and treatment facility, and home measurement data based on home measurement.

10. A terminal device used by a user, the terminal device comprising:
processing circuitry configured to
receive an input of disease information from the user;
access medical data about the user stored in a storage medium separated from a medical institution;
pick up medical data from the medical data stored, which is to be provided to the medical institution by the user in accordance with the disease information; and
propose the picked medical data to the user before the picked medical data is provided to the medical institution.

11. The terminal device according to claim 10, further comprising:
an input interface configured to input information received by the processing circuitry; and
an output interface configured to output information proposed by the processing circuitry to the user.

12. The terminal device according to claim 11, wherein the processing circuitry further receives an input of an authorized-provision-related personal health record (PHR) whose provision is authorized by the user within the medical data and provides information about the disease information that has been received and the authorized-provision-related PHR to the medical institution.

13. A medical information proposal device comprising:
processing circuitry configured to
receive information about the disease information from the terminal device according to claim 10 via a communication interface;
generate the medical data to be provided to the medical institution by the user from a provision target database on the basis of the information about the disease information that has been received; and
transmit the generated medical data to the terminal device via the communication interface.
